(19) United States
(12) Patent Application Publication
(10) Pub. No.: US 2022/0133807 A1
(43) Pub. Date: May 5, 2022

(54) COMPOSITIONS DERIVED FROM HUMAN AMNION CELLS & RELATED METHODS

(71) Applicant: AXOLOTL BIOLOGIX, INC, Flagstaff, AZ (US)

(72) Inventors: Robert G. Audet, Dracut, MA (US); Robert S. Kellar, Flagstaff, AZ (US)

(73) Assignee: AXOLOTL BIOLOGIX, INC., Flagstaff, AZ (US)

(21) Appl. No.: 17/570,524

(22) Filed: Jan. 7, 2022

Related U.S. Application Data
(63) Continuation-in-part of application No. 17/290,662, filed on Apr. 30, 2021.
(60) Provisional application No. 62/895,444, filed on Sep. 3, 2019.
(60) Provisional application No. 62/884,987, filed on Aug. 9, 2019.

(51) Int. Cl. A61K 35/50; A61P 17/02; A61P 17/14

(52) U.S. Cl. CPC A61K 35/50; A61P 17/14; A61P 17/02

(57) ABSTRACT

Methods of treating alopecia, connective tissue disease, or chronic skin wounds in a subject by administration of a therapeutically effective amount of a novel acellular human amnion-derived composition are disclosed. The novel acellular human amnion-derived composition is generally characterized as containing: one or more tissue-remodeling biomolecules, one or more proliferation biomolecules, one or more angiogenic biomolecules, one or more migration biomolecules, one or more anti-inflammatory biomolecules, and one or more anti-microbial biomolecules. Moreover, the acellular human amnion-derived composition is sterilized under conditions that preserve biological functionality and efficacy. Other features and characteristics of the treatment methods and compositions are described.

### CROSS-REFERENCE TO RELATED APPLICATIONS

This application is a continuation in part of U.S. Ser. No. 17/290,662, filed Apr. 30, 2021;

which is a 371 of PCT/US20/45664, filed Aug. 10, 2020;

which claims benefit of provisional application Ser. Nos. 62/895,444, filed Sep. 3, 2019 and 62/884,987, filed Aug. 9, 2019;

the entire contents of each of which is hereby incorporated by reference.

### TECHNICAL FIELD

The invention relates to compositions derived from human amnion cells, and more particularly, growth-factor and cytokine-rich fluids derived from human amnion cells which are useful to treat a variety of ailments; and methods for making and using the same.

### BACKGROUND ART

There is currently no regenerative therapy that can be used to: (i) alleviate pain associated with connective tissue disease (CTD), and more particularly, degenerative joint disease, (ii) protect tissue from degenerative joint disease, and (iii) regenerate joint tissue to restore bio-function at the affected joint.

### SUMMARY OF INVENTION

### Technical Problem

Osteoarthritis is a degenerative joint disease, wherein cartilage wears away gradually causing pain, dysfunction and/or disability. While common in the hands and spine, osteoarthritis may also affect the hips, knees, feet, ankles, shoulders, and adjacent soft tissues.

Total joint replacement surgery is the gold standard treatment in patients with severe end-stage symptomatic osteoarthritis who have failed to respond to nonpharmacologic and pharmacologic management and who have significant impairment in their quality of life due to OA.

Pharmaceuticals that are often used to help relieve pain associated with osteoarthritis include: acetaminophen, nonsteroidal anti-inflammatory drugs (NSAIDs), and duloxetine (CYMBALTA®). While these pharmacotherapies may help to reduce pain, they are not regenerative and ultimately the patient may require total joint replacement surgery.

There is a need for a regenerative therapy that can be used to treat osteoarthritis. In particular, there is a need for a novel biologic treatment which can: (i) alleviate pain associated with osteoarthritis, (ii) protect tissue from degenerative joint disease, and (iii) regenerate joint tissue to restore bio-function at the affected joint.

While this disclosure may explicitly describe osteoarthritis, each of these problems spans other ailments associated more generally with connective tissue disease (CTD). As such, the scope of the invention may be applicable to other ailments associated with CTD, and some related diseases which do not involve connected tissue, such as, for example, hair follicle arrest and chronic skin wounds.

### Solution to Problem

Disclosed is a fluid composition configured for local injection at a site of connective tissue disease, and more particularly, in accordance with one embodiment, at a site of degenerative joint disease. The fluid composition comprises a growth factor and cytokine-rich fluid that is derived from human amnion cells, which we refer to herein as an “acellular human amnion-derived fluid composition” or “fluid”. The fluid comprises biomolecules that, when administered to a subject, especially at a local site of connective tissue disease, may induce: (i) tissue remodeling; (ii) cellular proliferation and differentiation; (iii) angiogenesis; (iv) cell migration; (v) anti-inflammatory responses; and (vi) anti-microbial activity.

### Advantageous Effects of Invention

Delivery by intra-articular or peri-articular injection can present the fluid, including cytokines and growth factors thereof, to the site of the connective tissue disease, such as a degenerative joint disease, which immediately and efficiently serves to provide therapeutic benefit at the location of interest.

In degenerative joint disease and chronic skin wounds, the anti-inflammatory biomolecules present in the fluid function to reduce inflammation, thereby helping to relieve pain.

Growth factors supporting epithelial proliferation and differentiation, angiogenesis, and remodeling are present in the fluid and function to repair and restore soft tissue.

Other features and benefits will be appreciated by one having skill in the art upon a thorough review of the instant disclosure.

### BRIEF DESCRIPTION OF DRAWINGS

FIG. 1 is a flow chart representing a method for making a growth-factor and cytokine-rich fluid derived from human amnion cells.

FIG. 2 shows a comparison of tissue-remodeling biomolecules as determined from bio-assays for each of CdM1 and CdM2.

FIG. 3 shows a comparison of proliferation and differentiation biomolecules as determined from bio-assays for each of CdM1 and CdM2.

FIG. 4 shows a comparison of angiogenic biomolecules as determined from bio-assays for each of CdM1 and CdM2.

FIG. 5 shows a comparison of cell migration biomolecules as determined from bio-assays for each of CdM1 and CdM2.

FIG. 6 shows a comparison of anti-inflammatory biomolecules as determined from bio-assays for each of CdM1 and CdM2.

FIG. 7 shows a comparison of other biomolecules, including anti-microbial, osteogenesis, pro-apoptotic, pro-inflammatory, and other uncategorized regenerative biomolecules, as determined from bio-assays for each of CdM1 and CdM2.

FIG. 8 illustrates a clinical example of a non-healing wound (chronic skin wound), and subsequent treatment with an acellular human amnion-derived composition as-described herein.

FIG. 9 illustrates a clinical example of a fibular fracture treated with an acellular human amnion-derived composition as-described herein.

FIG. 10 illustrates a clinical example of degenerative joint disease, particularly ankle osteoarthritis, treated with an acellular human amnion-derived composition as-described herein.

FIGS. 11(A-D) illustrate the acellular human amnion-derived composition includes growth factors and cytokines produced by MSCs in culture.

FIG. 12A shows treatment with a human amnion derived composition as described herein increased hair follicle count compared to control.

FIG. 12B shows treatment with a human amnion derived composition as described herein increased hair follicle count per unit area compared to control.

FIG. 13A shows a pathology slide image of control treated skin.

FIG. 13B shows a pathology slide image of skin treated with a human amnion derived composition as described herein, epidermis is generated, and hair follicles are present.

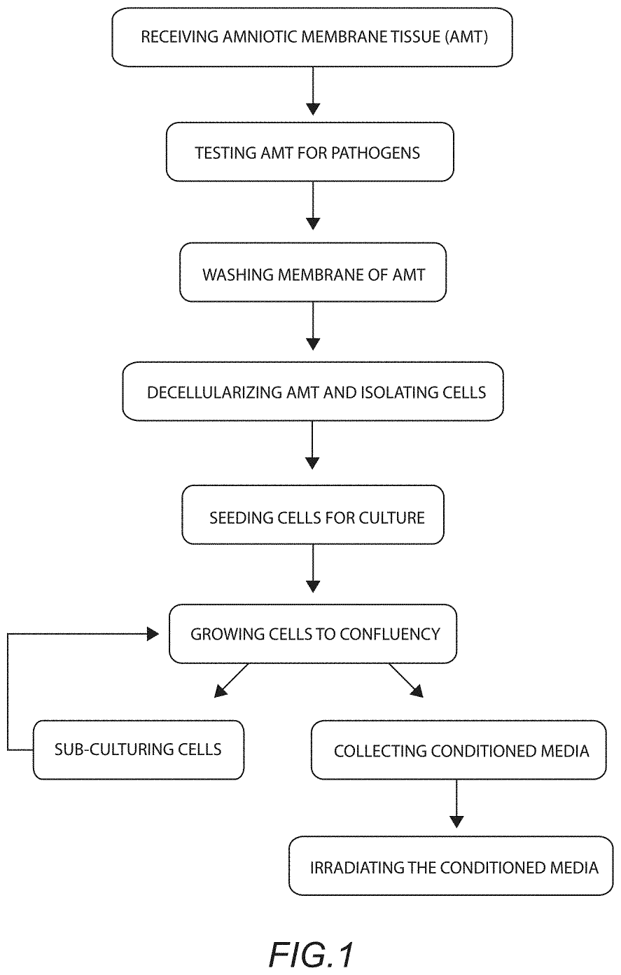

### DESCRIPTION OF EMBODIMENTS

Disclosed herein is an acellular human amnion-derived fluid composition which has been surprisingly discovered to comprise a unique combination of biomolecules, such as growth factors and cytokines, which aid in the repair and restoration of soft tissue, especially in soft tissue affected by connective tissue disease, and more particularly, in soft tissue affected by degenerative joint disease.

For purposes herein, “connective tissue disease” means any disease that affects the parts of the body that connect the structures of the body together.

For purposes herein, “degenerative joint disease”, also referred to as “osteoarthritis”, means a type of arthritis that occurs when flexible tissue at the ends of bones wears down.

For purposes herein, “chronic skin wound” means any wound that does not heal in an orderly set of stages and in a predictable amount of time the way most wounds do; wounds that do not heal within three months are often considered chronic.

In a general embodiment, the invention is directed to a novel acellular human amnion-derived fluid composition, and methods for making and using the same.

In one aspect, an acellular human amnion-derived composition is disclosed, which comprises: one or more tissue-remodeling biomolecules; one or more proliferation biomolecules; one or more angiogenic biomolecules; one or more migration biomolecules; one or more anti-inflammatory biomolecules; and one or more anti-microbial biomolecules; wherein the composition is irradiated to achieve an ambient temperature stable acellular fluid.

For purposes herein, “tissue-remodeling biomolecules” means biomolecules that are implicated in the reorganization or renovation of existing tissues. The one or more tissue-remodeling biomolecules may comprise: cystatin B (CSTB); cystatin C (CST3); plasminogen activator inhibitor-1 (PAI-1); matrix metallopeptidase 1 (MMP1); matrix metallopeptidase 13 (MMP13); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TIMP2); decorin (DCN); or a combination thereof.

For purposes herein, “proliferation biomolecules” means biomolecules that are implicated in the growth of new tissue. The one or more proliferation biomolecules may comprise: erb-b2 receptor tyrosine kinase 2 (ERBB2); dipeptidyl peptidase 4 (DPP4); epidermal growth factor receptor (EGFR); macrophage-colony stimulating factor (MCSF); activated leukocyte cell adhesion molecule (ALCAM); or a combination thereof.

For purposes herein, “angiogenic biomolecules” means biomolecules that are implicated in the formation of new blood vessels. The one or more angiogenic biomolecules may comprise: pentraxin 3 (PTX3); angiogenin (ANG); fms related tyrosine kinase 1 (FLT1); thrombospondin 1 (THBS1); urokinase-type plasminogen activator (uPA); transforming growth factor beta induced (TGFBI); or a combination thereof.

For purposes herein, “migration biomolecules” means biomolecules that are implicated in the movement of cells to specific locations for tissue formation, wound healing and immune responses. The one or more migration biomolecules may comprise: syndecan 4 (SDC4); neuronal cell adhesion molecule (NRCAM); dickkopf WNT signaling pathway inhibitor 3 (DKK3); angiotensinogen (AGT); or a combination thereof.

For purposes herein, “anti-inflammatory biomolecules” means biomolecules that are implicated in the reduction of inflammation. The one or more anti-inflammatory biomolecules may comprise: follistatin like 1 (FSTL1); galectin 1 (LGALS1); or a combination thereof.

For purposes herein, “anti-microbial biomolecules” means biomolecules that are implicated in the killing of microorganisms or inhibition of their growth. The one or more anti-microbial biomolecules may comprise: beta-2-microglobulin (B2M).

For purposes herein, “osteogenesis biomolecules” means biomolecules that are implicated in the formation of bone. The composition may further comprise one or more osteogenesis biomolecules. The one or more osteogenesis biomolecules may comprise: follistatin like 3 (FSTL3); growth differentiation factor 15 (GDF15); or a combination thereof.

For purposes herein, “pro-inflammatory biomolecules” means biomolecules that are implicated in the promotion of inflammation and related inducement of an immune response. The composition may further comprise one or more pro-inflammatory biomolecules. The one or more pro-inflammatory biomolecules may comprise: tumor necrosis factor receptor 1 (TNFR1).

For purposes herein, “pro-apoptotic biomolecules” means biomolecules that are implicated in promoting or causing apoptosis in cells. The composition may further comprise one or more pro-apoptotic biomolecules. The one or more pro-apoptotic biomolecules may comprise: Fas cell surface death receptor (FAS).

While certain examples of biomolecules are described, it should be understood that each biomolecule, while classified herein according to a particular function, may be alternatively classified as a different type of biomolecule where it has secondary function. For example, growth differentiation factor 15 (GDF15) is primarily an osteogenesis biomolecule; however, GDF15 has secondary function allowing it to be described as a tissue-remodeling biomolecule according to the knowledge and skill in the art.

In another aspect, a method for making an acellular human amnion-derived composition configured for therapeutic use is disclosed, the method comprises: obtaining amniotic membrane tissue; testing the amniotic membrane tissue for pathogens; washing the amniotic membrane tissue; manually removing blood-containing chorion tissue from the amniotic membrane tissue, decellularizing the amniotic membrane tissue with xeno-free enzymes; collecting cells from the decellularized amniotic membrane tissue; seeding the cells for culture into xeno-free media formulated for mesenchymal stem cells; growing the cells to a specified confluency; collecting conditioned media; and freezing the collected conditioned media; wherein the method further comprises: irradiating the frozen conditioned media.

The method may further comprise: freezing the collected conditioned media at −40° C. prior to irradiating the frozen conditioned media.

The method may further comprise: thawing the conditioned media; pooling one or more volumes of identical passages of the conditioned media from a common lot; aliquoting pooled conditioned media into desired volumes; and freezing the aliquots at −40° C.

The method may further comprise: subsequent to growing the cells to desired confluency, sub-culturing the cells and repeating the steps of: collecting conditioned media and irradiating the conditioned media obtained from the sub-cultured cells.

In yet another embodiment, a method for treating a subject suffering from connective tissue disease is disclosed, the method comprises: administering a therapeutically effective amount of an acellular human amnion-derived composition to soft tissue of the subject; whereby the subject is treated.

The method for treating a subject suffering from connective tissue disease is further distinguished wherein said acellular human amnion-derived composition comprises: one or more tissue-remodeling biomolecules; one or more proliferation biomolecules; one or more angiogenic biomolecules; one or more migration biomolecules; one or more anti-inflammatory biomolecules; and one or more anti-microbial biomolecules; wherein the composition is irradiated to render an acellular matrix. The connective tissue disease may comprise degenerative joint disease, other conditions which may benefit from the compositions and methods herein may include: hair follicle arrest and chronic skin wounds. Other connective tissue diseases, though not explicitly listed, may be similarly treated. In a preferred embodiment, the degenerative joint disease being treated comprises: ankle osteoarthritis.

The various embodiments of the invention will be better appreciated with the details as provided in the following examples with reference made to the related drawings.

### Example 1: Acellular Human Amnion-Derived Fluid Composition for Use in Soft Tissue Repair and Regeneration

### Tissue Preparation

Human placental tissue is obtained from a consenting donor in accordance with regulatory and other requirements. The tissue is placed in a sample container, and generally is suspended in natural fluid suspension. A swab is taken from the tissue at collection and tested for microbial contamination.

Serology is performed on donor blood serum for screening purposes.

If serology or 7-day culture results identify the presence of contamination or communicative diseases the membrane and all downstream cultures are destroyed.

### Cell Isolation & Membrane Decellularization

Pen-Strep antibiotic and mesenchymal stem cell culture media (culture media) were pre-warmed to room temperature. Three large sterile Erlenmeyer flasks were prepared for membrane washing with 200 mL of 1× Hank's Balanced Salt Solution (HBSS). The amniotic membrane was transferred aseptically into the first wash flask, closed with a sterile silicone stopper and placed on an orbital shaker for at least 20 minutes. After the first wash, the membrane was laid out on a sterile, stainless steel tray. With sterile gloves, the blood clots were manually rubbed or picked off of the membrane. Sections of the membrane where blood is trapped were untangled or excised until all visible blood clots were removed. Membrane was aseptically transferred to the second wash flask and 2 mL of 100× Pen-Strep antibiotic was added, the flask was placed on an orbital shaker for at least 20 minutes. During this incubation, three Erlenmeyer flasks were prepared for digestion, each with 100 mL of 1× TrypLE Select, 5 mM EDTA, and 1×HBSS.

The amnion was transferred to the first digestion flask for the preliminary TrypLE digest and incubated 10 minutes at 37° C., agitating the flask every 5 minutes. Following first digestion, the membrane was carefully moved to the second digestion flask, and incubated for 30 minutes at 37° C., agitating every 5 minutes. The first digest solution was properly disposed. After the second TrypLE digest was complete, the membrane was carefully moved to the third digestion flask and incubated for 30 minutes at 37° C., agitating every 5 minutes. The second TrypLE digest solution was properly disposed. After the third TrypLE digest was complete, 100 mL of 1×HBSS was added to the digest to dilute the TrypLE, and the flask was swirled to mix the solution. The membrane was carefully transferred to the third wash flask of 1×HBSS and the membrane swirled to dilute the TrypLE.

The solution from the third TrypLE digest was transferred into centrifuge tubes and centrifuged for 5 minutes at 200×g. The supernatant from each tube was carefully aspirated, leaving ˜0.5 mL supernatant above the pellet. The pellets were gently flicked to break them apart and triturated to resuspend them in the remaining supernatant. Resuspended cell pellets were pooled into a single 50 mL tube and 10 mL of cell culture media was added. The cell suspension was filtered through a 70-100 μm cell strainer into a fresh, sterile 50 ml tube. Cells were with a hemocytometer using Trypan blue to assess viability.

### Cell Seeding & Growth

Cells isolated from the amnion were triturated 10-20 times to produce a single cell suspension. Cells were seeded at approximately 10-30 million viable cells per T-25 flask. Additional culture media was added to the flasks, totaling 20 mL in a T25. One hundred microliters of 100× Pen-Strep was added to reach a final concentration of 0.5×. The flasks were incubated at 37° C. and 5% CO2. Every 2-3 days, or as needed, each flask should be inspected on an inverted microscope for culture health and confluence. If the culture is less than 60% confluent, the flask is returned to the incubator and until it is 60-80% confluent. The flasks were subcultured and the media was collected. If a flask is determined to be over-seeded, then the density may be adjusted in accordance with known techniques.

### Media Collection

Conditioned media (CdM) is collected for cultures that are to be subcultured, at a target confluence of 60-80% or when the cells are not to be expanded/sub-cultured further at 80-100% confluence. The CdM is aseptically transferred from the cell culture flask into one or more 50 mL conical tubes. Using a pipette, 42 mL of the fluid product is withdrawn from each flask to test for microbial contamination. The 50 mL conical tube(s) of CdM are frozen for storage.

The cell culture flask(s) are appropriately disposed unless they will be used for sub-culturing.

### Cell Subculturing

When a cell culture flask reaches target confluence and is ready to be subcultured, the CdM is collected as described above. The flask is rinsed with 1×HBSS and trypsinized by adding 1× TrypLE Select solution at, for example, 1 mL/T25 flask or 2 mL/T75 flask and incubated for 5-15 minutes at 37° C., until 80% or more of the cells have rounded up and are still adherent. Cells are gently dislodged and digestion halted by adding 3 volumes of culture media and gently triturating the suspension down the flask wall several times to create a single cell suspension. Cell suspension is transferred to a conical tube and centrifuged 5 minutes at 200×g. Supernatant is removed, and tube is gently flicked to disrupt the pellet. We added 1-2 mL of culture media and triturated the cell suspension to create a single cell suspension. Cells are counted with a hemocytometer, using Trypan blue to assess viability. Flasks are seeded at 10,000-20,000 cells/cm2 into suitable sized flasks to final volumes of 10-15 mL culture media/T25 and 20-30 mL/T75. Incubate at 37° C. and 5% CO2.

### Packaging & Sterilization

Each 50 mL conical tube of thawed CdM which has passed microbial testing is packaged for commercial distribution.

Using proper aseptic technique, the conditioned media from each conical tube is pipetted into the sterile cryovials. Each vial should receive the target volume of conditioned media with an additional 0.1 mL. Each conical tube should be pipetted into cryovials until there is only about 5 mL of conditioned media remaining. After vials are filled, the corresponding caps should be torqued to manufacturer's specifications.

The vials are subsequently irradiated, between 5 kGy and 50 kGy, and more preferably between 15 kGy and 22 kGy using e-beam radiation, or as otherwise appreciated by one having skill in the art. Alternatively, the vials may be sterilized by gamma irradiation, X-Ray, and/or sterile filtration. Sterility may be assessed by sterilization validation or by 14-day culture.

### Administration and Delivery

In preparation for use, a vial containing the fluid composition is optionally thawed (if frozen) and loaded in a syringe. Alternatively, the preparation may be provided in a pre-filled syringe. A physician administers the fluid composition by intra-articular or peri-articular injection at the site of degenerative joint disease. For chronic wounds, the fluid composition is injected within the wound bed and into the wound margins.

In some embodiments, the preparation is manufactured into a topical formulation as would be appreciated by one having skill in the art. The topical formulation may be applied to the skin of a patient.

### Example 2: Characterization of Amnion-Derived Fluid Composition

Two conditioned media samples herein (“CdM1” and “CdM2”) were obtained in accordance with the methods set forth in Example 1, above, and making use of human placental tissue from consenting donors. Note that “conditioned media” as used herein refers to the acellular human amnion-derived composition, which terms are interchangeable for purposes of this disclosure.

In an example, the control is the MSC culture media prior to culture with cells. Biomolecules within the resulting conditioned media samples were screened using conventional bioassays and immune assays and quantified as percent (%) above control.

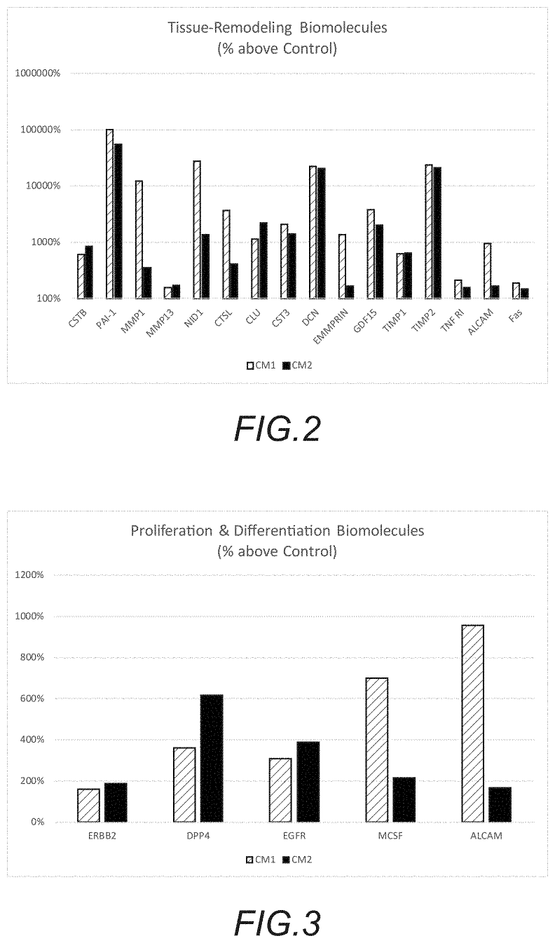

FIG. 2 shows a comparison of tissue-remodeling biomolecules as determined from bio-assays for each of CdM1 and CdM2. The tissue-remodeling biomolecules include: cystatin B (CSTB); cystatin C (CST3); plasminogen activator inhibitor-1 (PAI-1); matrix metallopeptidase 1 (MMP1); matrix metallopeptidase 13 (MMP13); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TIMP2); decorin (DCN); and growth differentiation factor 15 (GDF15). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these tissue-remodeling biomolecules at significant percent above control, suggesting that our method as outlined in Example 1, above, when practiced with human placental tissue, produces desirable biomolecules associated with tissue-remodeling.

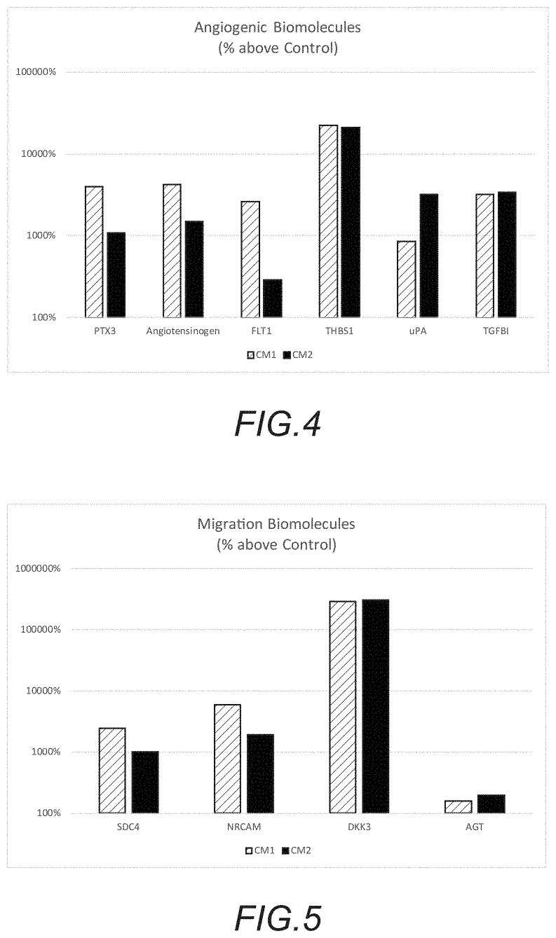

FIG. 3 shows a comparison of proliferation and differentiation biomolecules as determined from bio-assays for each of CdM1 and CdM2. The proliferation and differentiation biomolecules include: erb-b2 receptor tyrosine kinase 2 (ERBB2); dipeptidyl peptidase 4 (DPP4); epidermal growth factor receptor (EGFR); macrophage-colony stimulating factor (MCSF); and activated leukocyte cell adhesion molecule (ALCAM). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these proliferation and differentiation biomolecules at significant percent above control, suggesting that our method as outlined in Example 1, above, when practiced with human placental tissue, produces desirable biomolecules associated with proliferation and differentiation.

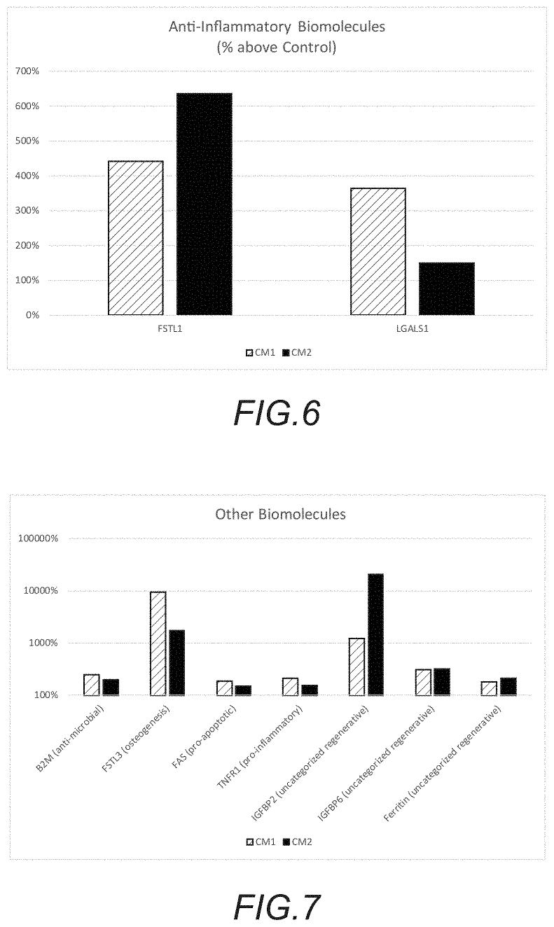

FIG. 4 shows a comparison of angiogenic biomolecules as determined from bio-assays for each of CdM1 and CdM2. The angiogenic biomolecules include: pentraxin 3 (PTX3); angiogenin (ANG); fins related tyrosine kinase 1 (FLT1); thrombospondin 1 (THBS1); urokinase-type plasminogen activator (uPA); and transforming growth factor beta induced (TGFBI). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these angiogenic biomolecules at significant percent above control, suggesting that our method as outlined in Example 1, above, when practiced with human placental tissue, produces desirable biomolecules associated with angiogenesis (blood vessel formation).

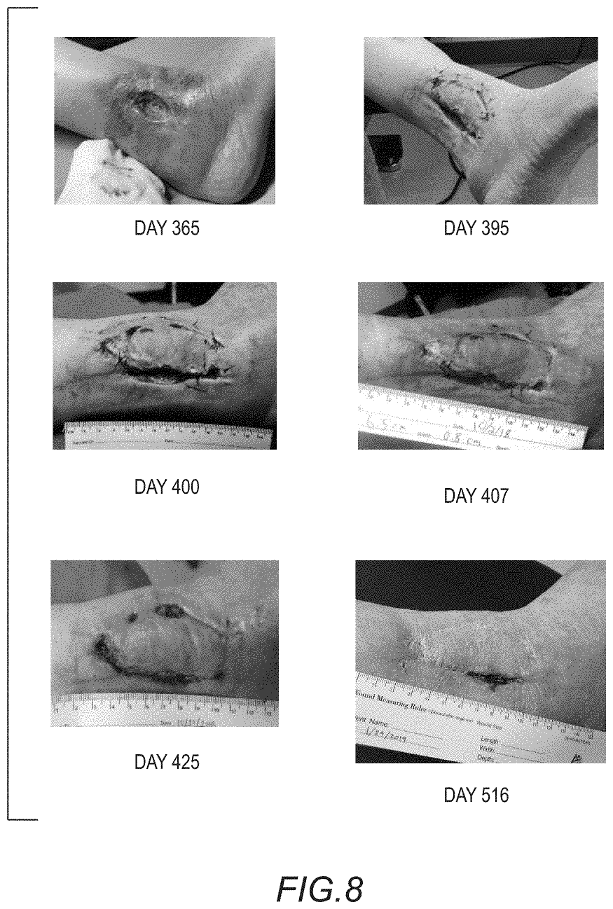

FIG. 5 shows a comparison of cell migration biomolecules as determined from bio-assays for each of CdM1 and CdM2. The cell migration biomolecules include: syndecan 4 (SDC4); neuronal cell adhesion molecule (NRCAM); dickkopf WNT signaling pathway inhibitor 3 (DKK3); and angiotensinogen (AGT). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these cell migration biomolecules at significant percent above control, suggesting that our method as outlined in Example 1, above, when practiced with human placental tissue, produces desirable biomolecules associated with cell migration.

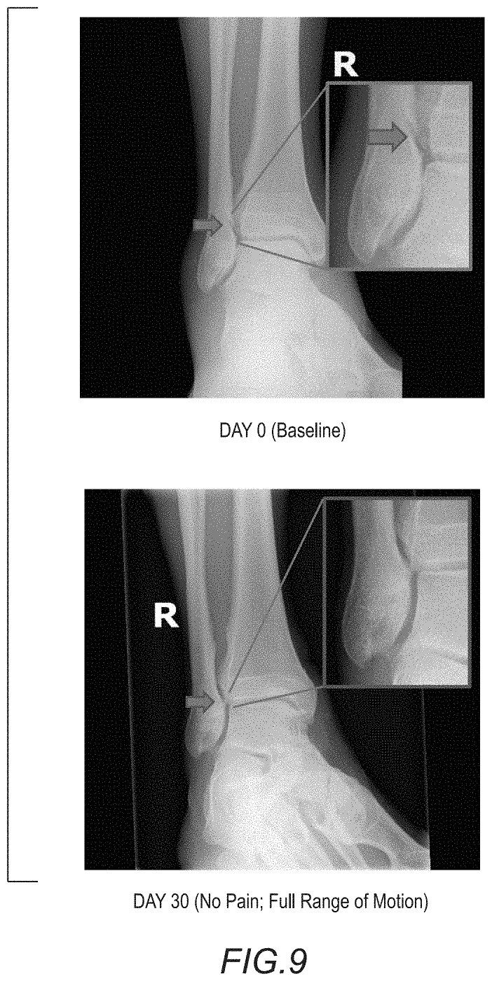

FIG. 6 shows a comparison of anti-inflammatory biomolecules as determined from bio-assays for each of CdM1 and CdM2. The anti-inflammatory biomolecules include: follistatin like 1 (FSTL1); and galectin 1 (LGALS1). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these anti-inflammatory biomolecules at significant percent above control, suggesting that our method as outlined in Example 1, above, when practiced with human placental tissue, produces desirable biomolecules associated with anti-inflammatory activity.

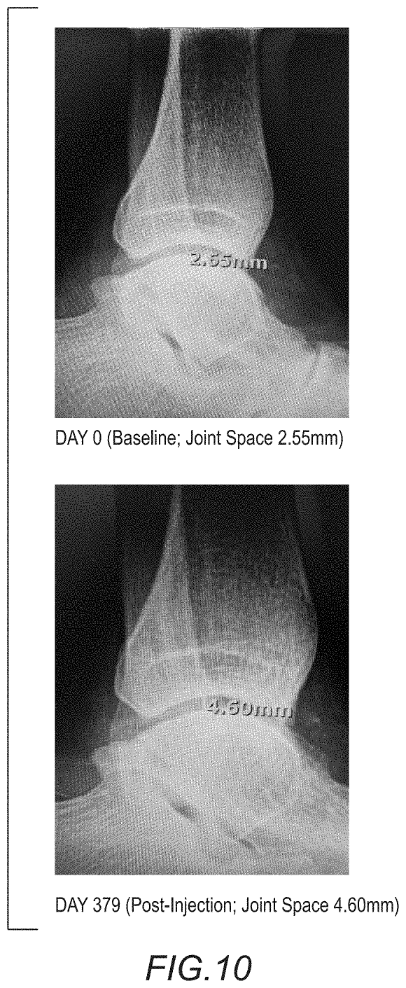

FIG. 7 shows a comparison of other biomolecules, including anti-microbial, osteogenesis, pro-apoptotic, pro-inflammatory, and other uncategorized regenerative biomolecules, as determined from bio-assays for each of CdM1 and CdM2. These biomolecules include: beta-2-microglobulin (B2M) which is an anti-microbial biomolecule; follistatin like 3 (FSTL3) which is an osteogenesis biomolecule; Fas cell surface death receptor (FAS) which is a pro-apoptotic biomolecule; tumor necrosis factor receptor 1 (TNFR1) which is a pro-inflammatory biomolecule; and other uncategorized regenerative biomolecules including: IGFBP2, IGFBP6, and Ferritin. One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced each of these biomolecules at significant percent above control, suggesting that our method as outlined in Example 1, above, when practiced with human placental tissue, produces desirable anti-microbial, osteogenesis, pro-apoptotic, pro-inflammatory, and other uncategorized regenerative biomolecules.

### Example 3: Acellular Human Amnion-Derived Composition for Chronic Wounds

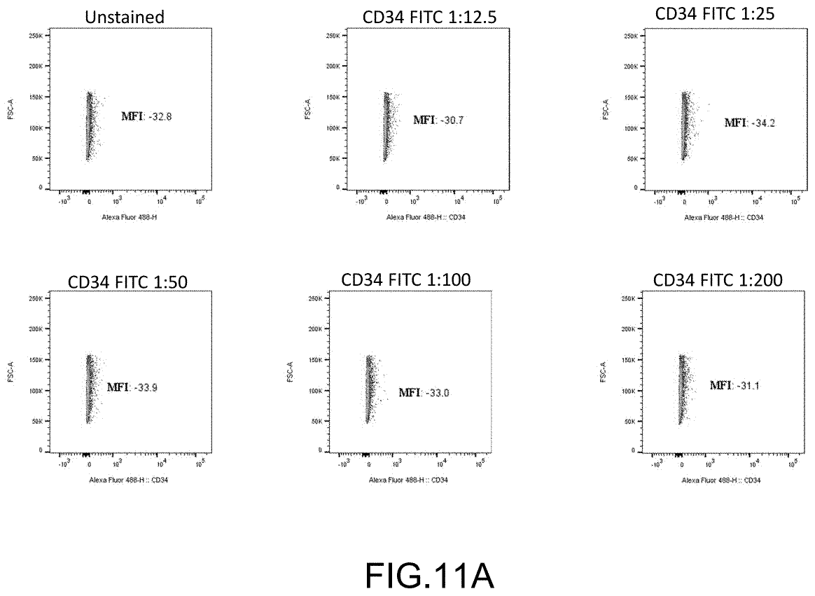

FIG. 8 depicts a clinical example of a forty-seven-year-old patient with broken ankle. Nearly one year after the injury (Day 365), the surgical site remained open. An acellular human amnion-derived composition, as described herein, was applied at various intervals (Day 365, Day 395, Day 400, Day 407, Day 425, and Day 516, respectively) and photographic data obtained. Within five months the wound was substantially healed. This example shows the clinical utility associated with the acellular human amnion-derived composition with respect to applications related to wound healing.

### Example 4: Acellular Human Amnion-Derived Composition for Fibular Fracture

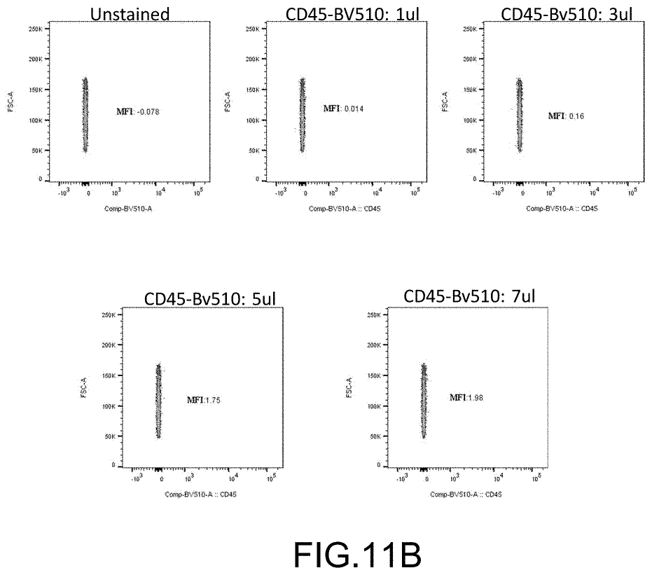

FIG. 9 depicts images obtained from a thirty-nine-year-old patient with a fibular fracture. A first image taken at Day 0 (baseline) shows the initial state of the fibular fracture. At this time, the patient complained of pain and restricted motion. The patient was treated with an acellular human amnion-derived composition as described herein. After thirty days (Day 30), the patient indicated the pain completely subsided and further demonstrated a full range of motion. An image was obtained, and the fracture is visibly healed.

### Example 5: Acellular Human Amnion-Derived Composition for Ankle Osteoarthritis

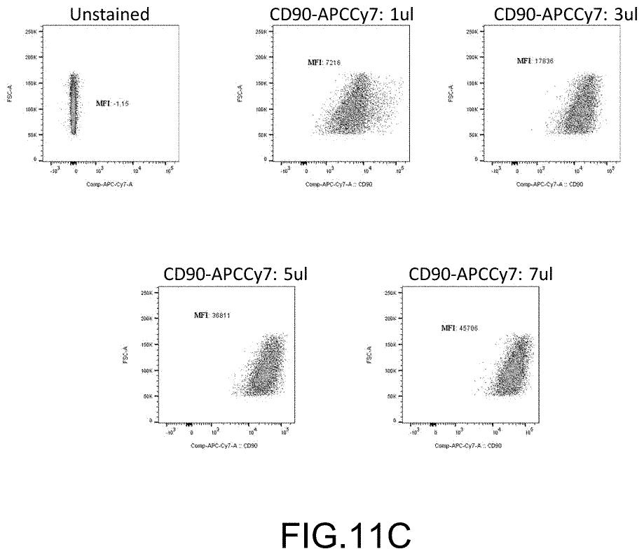

FIG. 10 depicts images obtained from a patient with ankle osteoarthritis. Pre-injection, a weight-bearing ankle x-ray image was obtained (Day 0; baseline), which revealed a joint space of about 2.55 mm. Regenerative therapy was achieved by later administering an acellular human amnion-derived composition as described herein. After about one year from the injury baseline (Day 379) another x-ray image was obtained post-injection, which reveals soft tissue regeneration in the joint space, which at that time measured 4.60 mm (just over 2.0 mm or about 80% improvement.

### Example 6: Mesenchymal Stromal Cells Expressing CD90 and CD105 After Multiple Passages

In certain embodiments, it is preferred to utilize mesenchymal stromal cells (MSCs) for up to four passages, or less. The reason for limiting use of MSCs to four passages is to ensure an optimal yield of growth factors and cytokines in the resulting acellular human amnion-derived composition.

Mesenchymal stromal cells are multipotent progenitor cells used in several cell therapies. MSCs are characterized by the expression of CD73, CD90, and CD105 cell markers, and the absence of CD34, CD45, CD11a, CD19, and HLA-DR cell markers. CD90 is a glycoprotein present in the MSC membranes and also in adult cells and cancer stem cells.

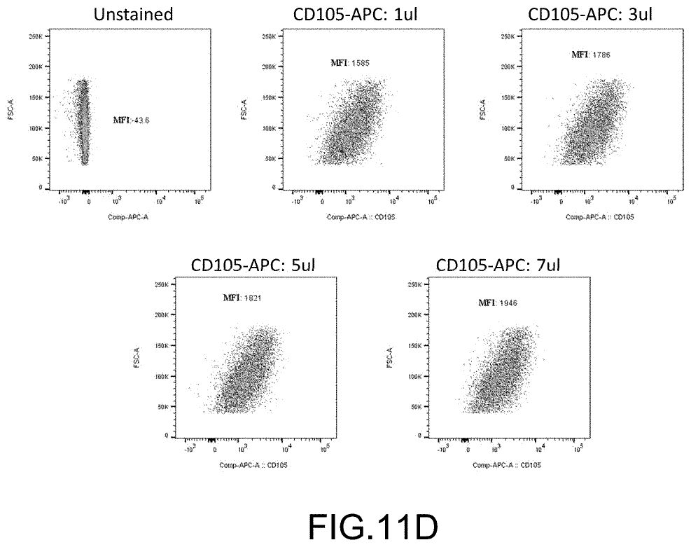

In this example, cells were tested after five (5) passages to identify expression of CD34; CD45; CD90; and CD105. Results of a standard bio assay reveal the MSCs produced according to the process described herein do not express CD34 and CD45, but do express CD90 and CD105. See FIGS. 11(A-D). Accordingly, the human amnion-derived composition includes cytokines and growth factors derived from MSCs.

### Example 7: Treatment of Alopecia

A method for treating alopecia (hair loss) in a subject comprises the step of administering to the subject a therapeutically effective amount of an acellular human amnion-derived composition comprising: one or more tissue-remodeling biomolecules selected from the group consisting of: cystatin B (CSTB), cystatin C (CST3), plasminogen activator inhibitor-1 (PAI-1), matrix metallopeptidase 1 (MMP1), matrix metallopeptidase 13 (MMP13), nidogen-1 (NID1), cathepsin L (CTSL), clusterin (CLU), extracellular matrix metalloproteinase inducer (EMMPRIN), TIMP metallopeptidase inhibitor 1 (TIMP1), TIMP metallopeptidase inhibitor 2 (TIMP2), decorin (DCN), or a combination thereof, one or more proliferation biomolecules selected from the group consisting of: erb-b2 receptor tyrosine kinase 2 (ERBB2), dipeptidyl peptidase 4 (DPP4), epidermal growth factor receptor (EGFR), macrophage-colony stimulating factor (MCSF), activated leukocyte cell adhesion molecule (ALCAM), or a combination thereof; one or more angiogenic biomolecules selected from the group consisting of: pentraxin 3 (PTX3), angiogenin (ANG), fms related tyrosine kinase 1 (FLT1), thrombospondin 1 (THBS1), urokinase-type plasminogen activator (uPA), transforming growth factor beta induced (TGFBI), or a combination thereof, one or more migration biomolecules selected from the group consisting of: syndecan 4 (SDC4), neuronal cell adhesion molecule (NRCAM), dickkopf WNT signaling pathway inhibitor 3 (DKK3), angiotensinogen (AGT), or a combination thereof; one or more anti-inflammatory biomolecules selected from the group consisting of: follistatin like 1 (FSTL1), galectin 1 (LGALS1), or a combination thereof, and one or more anti-microbial biomolecules including beta-2-microglobulin (B2M); whereby the subject is treated.

By way of example and not limitation, an acellular human amnion-derived composition was prepared according to the methods described in Example 1. The composition, referred to here as CdMx, was evaluated and it was determined to comprise: CSTB, CST3, PAI-1, MMP1, MMP13, NID1, CTSL, CLU, EMMPRIN, TIMP1, TIMP2, DCN, ERBB2, DPP4, EGFR, MCSF, ALCAM, PTX3, ANG, FLT1, THBS1, uPA, TGFBI, SDC4, NRCAM, DKK3, AGT, FSTL1, LGALS1, and B2M. See FIGS. 2-7.

Based on clinical observations and murine studies that demonstrated effects on hair regeneration, histological images were analyzed comparing hair regrowth and regeneration in CdMx treated wound margins and those to similar sized control wound margins. Additionally, preliminary data from growth factor panels examining CdMx was obtained. Analysis suggests that factors present in CdMx have proliferative and stimulating bioactivity that support hair follicle morphogenesis and/or support progression of the hair cycle. Of note, there are several other growth factors that may indirectly contribute to the regenerative hair process that can be investigated, as requested (for example, many growth factors could be linked to angiogenesis which is broadly supportive of hair regeneration).

The scope of these experiments include: (i) analyzing histological data from three control sites and four treated sites, counting follicular presence and total follicle count per unit area; (ii) using a historic Ray Biotech molecular panel of 440 growth factors (https://www.raybiotech.com/), 71 growth factors were selected based on higher expression relative to control media; and (iii) the 71 selected growth factors were reviewed for direct signaling implications in hair regeneration.

A total of seven wound sites were assessed for follicular presence after H & E staining. Using a blinded, de-identified image deck, counted by a non-biased researcher, the wound margins were explored for follicular presence. Once a count per wound margin was completed, a statistical assessment using standard t-Test was run on these data.

Each growth factor of the 71 selected growth factors was analyzed using a matrix of relevant search terms to evaluate direct roles in hair cycling. Of the 71 growth factors, 13 were determined to be highly relevant to hair regeneration.

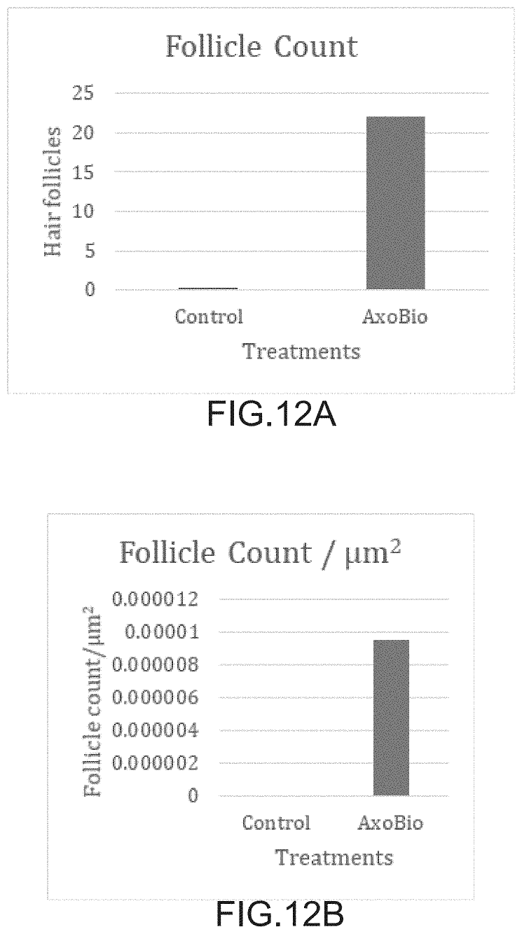

FIG. 12A shows hair follicle count for each of control- and CdMx-treated sites. The CdMx-treated sites showed a mean hair follicle count of twenty-two (22), whereas the control sites showed a mean hair follicle count of less than one (0.33). FIG. 12B shows density of hair follicle regeneration by way of follicle count per unit area. Here, the control showed zero follicles per square micrometer (no improvement), whereas the CdMx-treated site showed 9.56×10-6 follicles per μm2. The p-value was found to be 0.06 for both total follicular count per wound margin and for follicular count per unit area. These results indicate that CdMx composition is useful for treating alopecia (hair loss) due to hair follicle arrest.

### Example 8: Treatment of Skin Wounds

A method for treating a chronic skin wound in a subject comprises the step of administering to the subject a therapeutically effective amount of an acellular human amnion-derived composition comprising: one or more tissue-remodeling biomolecules selected from the group consisting of: cystatin B (CSTB), cystatin C (CST3), plasminogen activator inhibitor-1 (PAI-1), matrix metallopeptidase 1 (MMP1), matrix metallopeptidase 13 (MMP13), nidogen-1 (NID1), cathepsin L (CTSL), clusterin (CLU), extracellular matrix metalloproteinase inducer (EMMPRIN), TIMP metallopeptidase inhibitor 1 (TIMP1), TIMP metallopeptidase inhibitor 2 (TIMP2), decorin (DCN), or a combination thereof, one or more proliferation biomolecules selected from the group consisting of: erb-b2 receptor tyrosine kinase 2 (ERBB2), dipeptidyl peptidase 4 (DPP4), epidermal growth factor receptor (EGFR), macrophage-colony stimulating factor (MCSF), activated leukocyte cell adhesion molecule (ALCAM), or a combination thereof; one or more angiogenic biomolecules selected from the group consisting of: pentraxin 3 (PTX3), angiogenin (ANG), fms related tyrosine kinase 1 (FLT1), thrombospondin 1 (THBS1), urokinase-type plasminogen activator (uPA), transforming growth factor beta induced (TGFBI), or a combination thereof, one or more migration biomolecules selected from the group consisting of: syndecan 4 (SDC4), neuronal cell adhesion molecule (NRCAM), dickkopf WNT signaling pathway inhibitor 3 (DKK3), angiotensinogen (AGT), or a combination thereof; one or more anti-inflammatory biomolecules selected from the group consisting of: follistatin like 1 (FSTL1), galectin 1 (LGALS1), or a combination thereof, and one or more anti-microbial biomolecules including beta-2-microglobulin (B2M); whereby the subject is treated.

By way of example and not limitation, an acellular human amnion-derived composition was prepared according to the methods described in Example 1. The composition, referred to here as CdMx, was evaluated and it was determined to comprise: CSTB, CST3, PAI-1, MMP1, MMP13, NID1, CTSL, CLU, EMMPRIN, TIMP1, TIMP2, DCN, ERBB2, DPP4, EGFR, MCSF, ALCAM, PTX3, ANG, FLT1, THBS1, uPA, TGFBI, SDC4, NRCAM, DKK3, AGT, FSTL1, LGALS1, and B2M. See FIGS. 2-7.

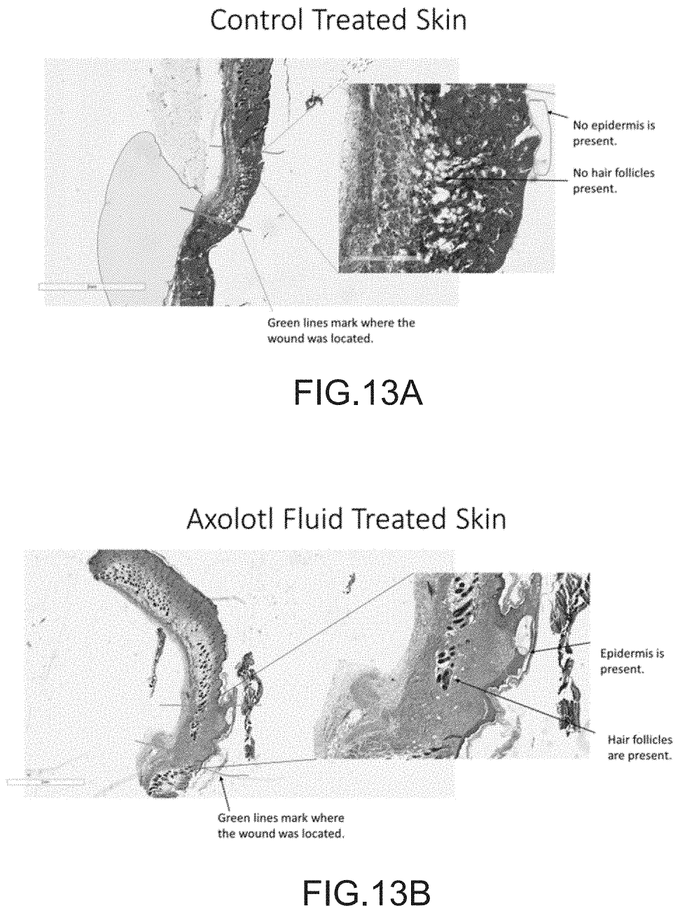

A conventional mouse model for wounds was utilized, with control- and CdMx-treatments, to assess, inter alia, wound healing. FIG. 13A shows an image obtained from a tissue section associated with control-treated skin. Here, there are visibly no epidermis and no hair follicles present in the sample. The color image includes green lines demarking where the wound was positioned. Turning now to FIG. 13B, an image obtained from a tissue section associated with CdMx-treated skin. Here, similar green lines are shown for locating position of the wound. Note that epidermis and hair follicles are present. Thus, the CdMx-treated site showed improved wound healing, epidermal and hairfollicle regeneration.

### Example 9: Molecular Panels

As discussed above, a molecular panel was obtained to investigate growth factors within the CdMx composition that contribute to hair and skin regeneration. Results indicate presence of at least thirteen (13) growth factors of interest, namely:

TABLE 1
Molecular Panel Growth Factors of Interest
for Hair and Skin Regeneration

| | |
|---|---|
| Growth Factor | Elevated % vs. Control (two Panels) |

| | | |
|---|---|---|
| Cystatin M/E | 140% | 297% |
| Angiogenin | 4225% | 1482% |
| PAI-1 | 100,730% | 55,697% |
| POSTN | 9,086% | 100% |
| GAL1 | 365% | 150% |
| DCN | 22,402% | 20,550% |
| EGF-R | 307% | 387% |
| HGF | 190% | 100% |
| TIMP-1 | 633% | 643% |
| TIMP-2 | 23,679% | 21,292% |
| Leptin | 139% | 151% |
| Cathepsin L | 3,645% | 415% |
| Clusterin | 1,132% | 2,178% |

Each of these growth factors is associated with hair and skin regeneration in the literature. The CdMx composition comprising these growth factors, combined with the experimental results related to hair and skin regeneration in mice, suggests that the CdMx composition is uniquely formulated with growth factors that promote hair and skin regeneration.

Table 2 shows a range between low and high concentrations of several growth factors as measured in multiple batches of human amnion derived composition and tested for efficacy. Therefore, a therapeutically effective amount of the human amnion derived composition may comprise an amount of the various growth factors (in pg/mL) within the range of low and high concentrations as disclosed.

TABLE 2
Example Concentration of Therapeutic Composition

| | LOW Conc. | High Conc. |
|---|---|---|
| Growth Factor | (pg/mL) | (pg/mL) |
| Cystatin B (CSTB) | 179 | 354 |
| Cystatin C (CST3) | 1483 | 2823 |
| Plasminogen activator inhibitor 1 (PAI-1) | 1753 | 3029 |
| Matrix metallopeptidase 1 (MMP1) | 61 | 2711 |
| Nidogen (NID1) | 297 | 5353 |
| Cathepsin L (CTSL) | 74 | 373 |
| Clusterin (CLU) | 3341 | 7533 |
| Extracellular matrix metalloproteinase | 29 | 332 |
| inducer (EMMPRIN) | | |
| Tissue inhibitor of metallopeptidase 1 | 1512 | 1921 |
| (TIMP1) | | |
| Tissue inhibitor of metallopeptidase 2 | 2436 | 3728 |
| (TIMP2) | | |
| Decorin (DCN) | 820 | 1053 |

### INDUSTRIAL APPLICABILITY

The invention is applicable to the medical industry as it encompasses a biologic conditioned media, namely, an acellular human amnion-derived composition, which is useful as a therapeutic for soft tissue repair and remodeling, especially that which is desired in response to hair and skin regeneration.

What is claimed is:

1. A method of treating alopecia in a subject, comprising:
administering to the subject a therapeutically effective amount of an acellular human amnion-derived composition comprising:
one or more tissue-remodeling biomolecules selected from the group consisting of: cystatin B (CSTB), cystatin C (CST3), plasminogen activator inhibitor-1 (PAI-1), matrix metallopeptidase 1 (MMP1), matrix metallopeptidase 13 (MMP13), nidogen-1 (NID1), cathepsin L (CTSL), clusterin (CLU), extracellular matrix metalloproteinase inducer (EMMPRIN), TIMP metallopeptidase inhibitor 1 (TIMP1), TIMP metallopeptidase inhibitor 2 (TIMP2), decorin (DCN), or a combination thereof;
one or more proliferation biomolecules selected from the group consisting of: erb-b2 receptor tyrosine kinase 2 (ERBB2), dipeptidyl peptidase 4 (DPP4), epidermal growth factor receptor (EGFR), macrophage-colony stimulating factor (MCSF), activated leukocyte cell adhesion molecule (ALCAM), or a combination thereof,
one or more angiogenic biomolecules selected from the group consisting of: pentraxin 3 (PTX3), angiogenin (ANG), fms related tyrosine kinase 1 (FLT1), thrombospondin 1 (THBS1), urokinase-type plasminogen activator (uPA), transforming growth factor beta induced (TGFBI), or a combination thereof;
one or more migration biomolecules selected from the group consisting of: syndecan 4 (SDC4), neuronal cell adhesion molecule (NRCAM), dickkopf WNT signaling pathway inhibitor 3 (DKK3), angiotensinogen (AGT), or a combination thereof,
one or more anti-inflammatory biomolecules selected from the group consisting of: follistatin like 1 (FSTL1), galectin 1 (LGALS1), or a combination thereof, and
one or more anti-microbial biomolecules including beta-2-microglobulin (B2M);
whereby the subject is treated.

2. A method of treating connective tissue disease in a subject, comprising:
administering to the subject a therapeutically effective amount of an acellular human amnion-derived composition comprising:
one or more tissue-remodeling biomolecules selected from the group consisting of: cystatin B (CSTB), cystatin C (CST3), plasminogen activator inhibitor-1 (PAI-1), matrix metallopeptidase 1 (MMP1), matrix metallopeptidase 13 (MMP13), nidogen-1 (NID1), cathepsin L (CTSL), clusterin (CLU), extracellular matrix metalloproteinase inducer (EMMPRIN), TIMP metallopeptidase inhibitor 1 (TIMP1), TIMP metallopeptidase inhibitor 2 (TIMP2), decorin (DCN), or a combination thereof;
one or more proliferation biomolecules selected from the group consisting of: erb-b2 receptor tyrosine kinase 2 (ERBB2), dipeptidyl peptidase 4 (DPP4), epidermal growth factor receptor (EGFR), macrophage-colony stimulating factor (MCSF), activated leukocyte cell adhesion molecule (ALCAM), or a combination thereof,
one or more angiogenic biomolecules selected from the group consisting of: pentraxin 3 (PTX3), angiogenin (ANG), fms related tyrosine kinase 1 (FLT1), thrombospondin 1 (THBS1), urokinase-type plasminogen activator (uPA), transforming growth factor beta induced (TGFBI), or a combination thereof;
one or more migration biomolecules selected from the group consisting of: syndecan 4 (SDC4), neuronal cell adhesion molecule (NRCAM), dickkopf WNT signaling pathway inhibitor 3 (DKK3), angiotensinogen (AGT), or a combination thereof,
one or more anti-inflammatory biomolecules selected from the group consisting of: follistatin like 1 (FSTL1), galectin 1 (LGALS1), or a combination thereof, and
one or more anti-microbial biomolecules including beta-2-microglobulin (B2M);
whereby the subject is treated.

3. A method of treating a chronic skin wound in a subject, comprising:
administering to the subject a therapeutically effective amount of an acellular human amnion-derived composition comprising:
one or more tissue-remodeling biomolecules selected from the group consisting of: cystatin B (CSTB), cystatin C (CST3), plasminogen activator inhibitor-1 (PAI-1), matrix metallopeptidase 1 (MMP1), matrix metallopeptidase 13 (MMP13), nidogen-1 (NID1), cathepsin L (CTSL), clusterin (CLU), extracellular matrix metalloproteinase inducer (EMMPRIN), TIMP metallopeptidase inhibitor 1 (TIMP1), TIMP metallopeptidase inhibitor 2 (TIMP2), decorin (DCN), or a combination thereof;
one or more proliferation biomolecules selected from the group consisting of: erb-b2 receptor tyrosine kinase 2 (ERBB2), dipeptidyl peptidase 4 (DPP4), epidermal growth factor receptor (EGFR), macrophage-colony stimulating factor (MCSF), activated leukocyte cell adhesion molecule (ALCAM), or a combination thereof,
one or more angiogenic biomolecules selected from the group consisting of: pentraxin 3 (PTX3), angiogenin (ANG), fms related tyrosine kinase 1 (FLT1), thrombospondin 1 (THBS1), urokinase-type plasminogen activator (uPA), transforming growth factor beta induced (TGFBI), or a combination thereof;
one or more migration biomolecules selected from the group consisting of: syndecan 4 (SDC4), neuronal cell adhesion molecule (NRCAM), dickkopf WNT signaling pathway inhibitor 3 (DKK3), angiotensinogen (AGT), or a combination thereof,
one or more anti-inflammatory biomolecules selected from the group consisting of: follistatin like 1 (FSTL1), galectin 1 (LGALS1), or a combination thereof, and
one or more anti-microbial biomolecules including beta-2-microglobulin (B2M);
whereby the subject is treated.

4. The method of claim 1, wherein the composition is sterilized by irradiation.

5. The method of claim 4, wherein the composition is sterilized while in a frozen state.

6. The method of claim 2, wherein the composition is sterilized by irradiation.

7. The method of claim 6, wherein the composition is sterilized while in a frozen. state.

8. The method of claim 3, wherein the composition is sterilized by irradiation.

9. The method of claim 8, wherein the composition is sterilized while in a frozen.
